(19) United States
(12) Patent Application Publication
(10) Pub. No.: US 2022/0220059 A1
(43) Pub. Date: Jul. 14, 2022

(54) PROCESS FOR CRYSTALLIZATION OF HIGH PURITY LUTEIN ESTERS FROM MARIGOLD EXTRACTS

(71) Applicant: KEMIN INDUSTRIES, INC., Des Moines, IA (US)

(72) Inventor: Frederick Khachik, Urbandale, IA (US)

(21) Appl. No.: 17/575,227

(22) Filed: Jan. 13, 2022

Related U.S. Application Data
(60) Provisional application No. 63/137,491, filed on Jan. 14, 2021.

(51) Int. Cl. C07C 67/56; B01D 11/04; B01D 11/02; B01D 39/04; B01D 39/20; B01D 9/00

(52) U.S. Cl. CPC C07C 67/56; B01D 11/0492; B01D 11/028; C07B 2200/13; B01D 39/20; B01D 9/0045; B01D 9/0059; B01D 39/04

(57) ABSTRACT

The present invention relates to a novel process for crystallizing high purity lutein and zeaxanthin esters from marigold oleoresin. In another aspect, the present invention relates to the removal of soft and hard waxes by adsorption on diatomaceous earth or other filter aid using acetone or other organic solvent. Another aspect of the present invention relates to achieving high-purity lutein and zeaxanthin esters, including purity levels that are about 90 to 95%.

### CROSS-REFERENCE TO RELATED APPLICATIONS

This application claims the benefit of priority to U.S. Provisional Patent Application No. 63/137,491, filed Jan. 14, 2021, entitled “PROCESS FOR CRYSTALLIZATION OF HIGH PURITY LUTEIN ESTERS FROM MARIGOLD EXTRACTS,” the entire disclosure of which is hereby incorporated by reference in its entirety.

### BACKGROUND OF THE INVENTION

The present invention relates generally to a process for isolating and purifying lutein esters from marigold oleoresin.

Marigold oleoresin is an extract of marigold flowers with a total carotenoid content of 15-17% that consists of a mixture of lutein (94%) and zeaxanthin fatty acid esters (6%). It has been shown that various lots of this oleoresin contain significant amounts of cis-isomers of lutein (22-24%) accompanied by much lower levels of cis-zeaxanthins (4-5%).

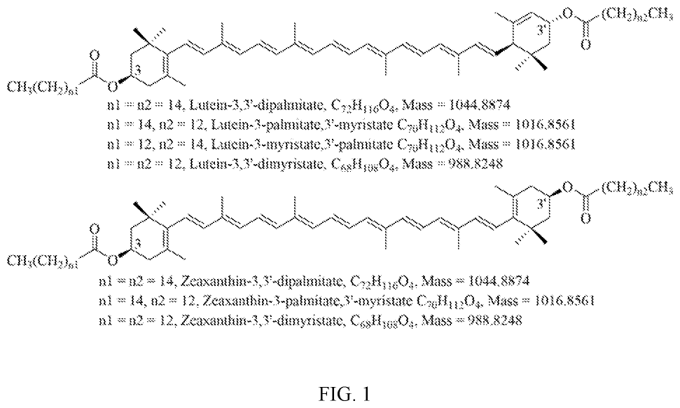

There are numerous reports on extraction and isolation of food carotenoids with nutritional significance from plants on large commercial scales and their formulation into dietary supplements. While in most fruits and vegetables lutein and zeaxanthin exist in their unesterified form, in marigold flowers these carotenoids are esterified with palmitic and myristic acids as well as their cross esters¹⁻⁴. Marigolds contain minor quantities (4-6%) of (3R,3′R)-zeaxanthin palmitate and myristate that are regioisomers of lutein palmitate and myristate. The chemical structures of all possible lutein esters (L-esters) and zeaxanthin esters (Z-esters) in marigold flowers are shown in FIG. 1.

The extracts from marigolds are saponified to transform lutein and zeaxanthin fatty acid esters to their corresponding dihydroxycarotenoids. In an earlier internal white paper (WP-19-574, see attached), the inventors previously established that hexane extracts of marigold flowers (marigold oleoresin) with 15-17% total carotenoids, consist of 66-68% all-trans-lutein that is accompanied by 22-24% of its cis-stereoisomers. Similarly, marigold oleoresin contains 5-6% of all-trans-zeaxanthin and 4-5% of its cis-stereoisomers. Since commercial production of unesterified lutein and zeaxanthin from this oleoresin by Kemin in 1996, marigold oleoresin has been widely used for commercial production of purified lutein and zeaxanthin esters by eliminating the saponification step.

The isolation of lutein fatty acid esters (L-esters) from plant materials, particularly marigold oleoresin, containing lutein esters was first published by Philip in 1977⁵. This was accomplished by solubilizing marigold oleoresin in hot alcohols such as methanol, ethanol, isopropanol, 1-propanol, and 1-butanol followed by cooling the resulting solution to crystallize lutein fatty acid esters in 51% purity and the remaining consisted of fats and triglycerides. In another patented process reported by Levy, concentrated extracts of xanthophyll esters were similarly crystallized with alcohols⁶. In 2007, Madhavi et al. also reported on crystallization of lutein esters from marigold oleoresin using a mixture of 1-propanol and ethanol⁷. In 2013, Wu et al. also employed C₂-C₄ alcohols to affect crystallization of lutein esters from extracts of marigold flowers⁸. In 2003 Kumar reported the crystallization of lutein and zeaxanthin fatty acid esters by employing a ketonic solvent such as 2-propanone (acetone), 2-butanone, 2-pentanone, or their mixtures⁹. According to this process, impurities in marigold oleoresin were solubilized in acetone that resulted in an enriched xanthophyll esters concentrate in 10-14% yield with lutein esters content of 46-64%. In another process, Sadano et al. subjected marigold oleoresin with 16.9% lutein ester content to supercritical fluid extraction using carbon dioxide at 50-60° C. to obtain an extract with increased purity of lutein esters that were subsequently dissolved in hot acetone and filtered. Upon cooling the acetone solutions, the extracts were enriched in 23.8-32.9 wt. % of lutein esters^{10,11}. In another patented process by Ornelas-Cravioto and Hernandez, the purification of lutein esters was accomplished with n-hexane that was used to solubilize marigold oleoresin and the mixture was then allowed to settle to partially crystallize lutein esters¹². According to this procedure, the upper phase was removed by decantation and the crystalline lutein esters were then washed with n-hexane four times prior to filtration. The high purity trans-lutein esters obtained according to a patented process by Ornelas-Cravito has been formulated into a colloidal solution employing edible oils¹³. In 2006, Xu et al. reported a purification process for xanthophyll fatty acid ester and xanthophylls by microporous adsorptive resin column chromatography¹⁴.

Combination of hexane and dichloromethane has also been employed by Chen et al. to obtain high purity lutein esters¹⁵. Other related patented processes have employed combination of ethyl acetate and alcohols for purification of lutein esters¹⁶⁻²¹. Several other patented process for purification of lutein esters have employed supercritical fluid extraction of marigold meals with carbon dioxide²²⁻²⁴. Finally, in a most recent process, Lian et al. employed mixed solvents for preparing high-purity lutein esters²⁵. This preparation involved: a) dissolving the lutein extract, mixing solvent, stirring to obtain a solution; wherein the mixed solvent lutein extract selected from group consisting of ethyl acetate, isopropanol, acetone, n-hexane, petroleum ether, and n-butanol.

After screening combination of several solvents for crystallization of lutein and zeaxanthin esters from marigold oleoresin that were used with the previously patented processes, several major problems were discovered. These were low purity and yield of lutein esters and unsuccessful removal of waxes and residual solvents from the crystallized esters by conventional drying processes. In addition, the use of high boiling solvent does not allow complete removal of solvents from the isolated lutein esters. This is because typical drying process that involves heating under vacuum cannot be used due to the low melting point of lutein esters.

Therefore, a novel process was developed by employing acetone as a single crystallization solvent to afford lutein and zeaxanthin esters in high purity with complete removal of residual solvent.

For these and other reasons, there is a need for the present invention.

### SUMMARY OF THE INVENTION

The present invention relates to methods of crystallizing lutein esters (L-esters) and zeaxanthin esters (Z-esters) from marigold oleoresin in high yield and purity. The marigold oleoresin is first solubilized in acetone or other appropriate organic solvent to form a mixture which is then filtered through Celite® 545 (Sigma-Aldrich cat # 22140), diatomaceous earth (such as Fisher Scientific cat # S25298) or other appropriate filter medium. During the filtration process, the soft and hard waxes in the marigold oleoresin are adsorbed onto the filter medium. The filtrate, containing organic solvent and the lutein esters and zeaxanthin esters, is next stirred to form crystals of lutein esters and zeaxanthin esters that are collected by filtration and dried. This second filtrate contains mostly the cis-isomers of lutein esters and zeaxanthin esters in organic solvent. The organic solvent in the second filtrate is then evaporated or otherwise removed to obtain an oleoresin that contains cis-isomers of lutein esters and zeaxanthin esters.

The unique process of the invention provides lutein esters that have a wide range of purity in the range of 60-95% and have been prepared in 90-95% purity. The lutein esters obtained by this process may be used as a dietary supplement or in the production of lutein and/or in partial synthesis of β-cryptoxanthin according to published procedures (F. Khachik, U.S. Pat. No. 9,725,411 B2, Aug. 8, 2017; F. Khachik, Archives Biochemistry & Biophysics, 2018, 653, 107-112). The process may further be expedited following the solubilization step by seeding the organic solvent solution with small amounts of crystalline trans-(L+Z)-esters to provide uniform and rapid crystallization of carotenoid esters. In addition, the by-product of organic solvent crystallization (the second filtrate) containing significant amount of (trans+cis)-(lutein+zeaxanthin)-esters can be potentially used in the production of alternative carotenoid products.

### DETAILED DESCRIPTION OF THE DRAWINGS

FIG. 1 illustrates the structures of all possible lutein esters and zeaxanthin esters in marigold flowers.

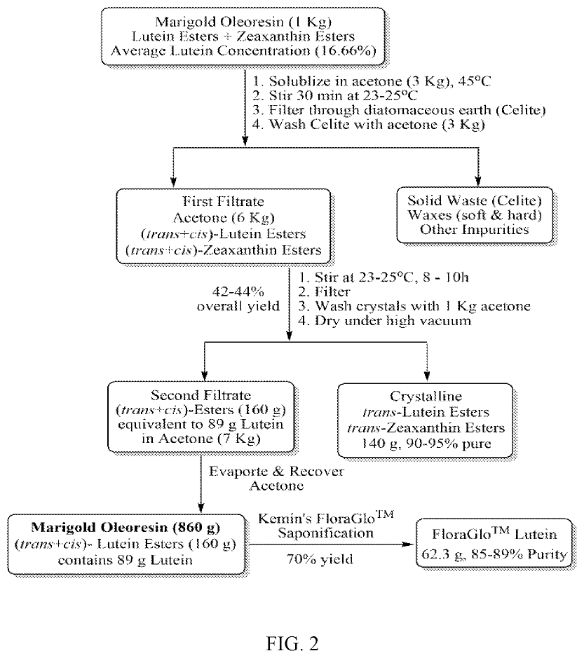

FIG. 2 is a flow chart illustrating the conversion of 1 Kg of marigold oleoresin to crystalline lutein esters.

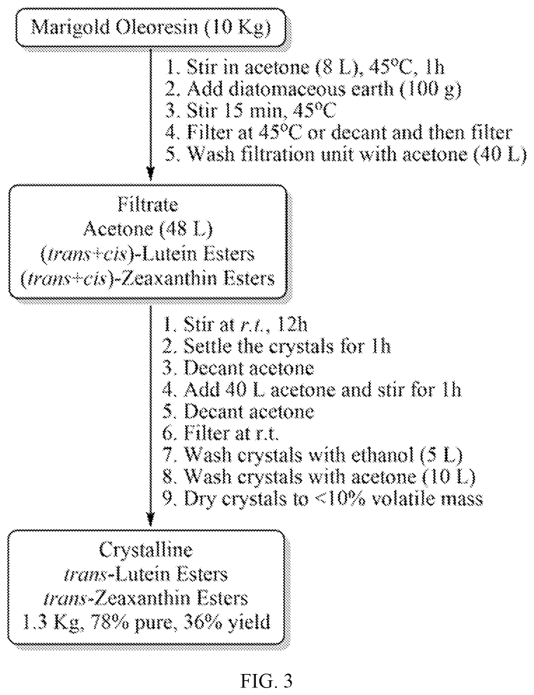

FIG. 3 is a flow chart illustrating the conversion of 10 Kg of marigold oleoresin to crystalline lutein esters.

### DETAILED DESCRIPTION OF THE PREFERRED EMBODIMENT

The present inventor has unexpectedly determined that highly pure lutein esters can be obtained through the removal of soft and hard waxes from marigold oleoresin by adsorption on diatomaceous earth or other filtering material.

As noted, the method of the invention involves the crystallization of lutein and zeaxanthin esters in high purity from marigold oleoresin. Marigold oleoresin is a dark brown, viscous liquid extracted from marigold which is rich in lutein esters (L-esters) that also contains minor quantities of zeaxanthin esters (Z-esters). Lutein obtained from saponification of marigold oleoresin is a natural food colorant used in beverages, confectionery products, dairy products, cereals, eggs, sauces, desserts, processed fruits, and frozen products, and is widely used in the pharmaceutical and dietary supplement industry due to its medical attributes. This yellow pigment is well known for its antioxidant and anti-cancer activities and further has antiseptic properties. It is used in animal feed, particularly in poultry for imparting yellow color to egg yolks.

In general, the marigold flower is harvested and the petals ideally separated from the stalk prior to the extraction process. After extraction of marigold flowers with an organic solvent, the solvent is removed to obtain an oleoresin that is enriched in lutein esters and minor quantities of zeaxanthin esters. The marigold oleoresin is then solubilized in a solvent system to provide crystallization of the lutein esters. Several solvent systems are appropriate for this purpose so long as they are capable of solubilizing the oleoresin and affect crystallization, including a combination of alcohols and ethyl acetate (EtOAc), such as 1-propanol (PrOH) and EtOAc, PrOH/acetone, and EtOAc/acetone. The marigold oleoresin may also be solubilized in acetone alone. The solvents may be used in any amounts and/or ratios sufficient to solubilize the oleoresins as may be readily understood by persons skilled in the art. If a solvent system such as a combination of alcohols and ethyl acetate is used, the weight ratio of PrOH:EtOAc:oleoresin may range from about 0.8:0.4:1.0 to 3:1:1. If the solvent system is consisted of PrOH and acetone, the weight ratio of PrOH:acetone:oleoresin is in the range of 1:3:1 to 3:1:1. Similarly, in the case of acetone and EtOAc the weight ratio of acetone:EtOAc:oleoresin is in the range of 4.5:1.5:1 to 3.0:0.5:1.0 or alternatively the ratio may vary from 2.5:0.5:1.0 to 1:1:1. When acetone is used as a single solvent the weight ratio of acetone to oleoresin may range from 5:1 to 2:1, preferably 3:1.

The mixture is preferably heated to a temperature of at least 40° C. to solubilize the oleoresins, then stirred at ambient temperatures (23-25° C.) for a time period of around 30 minutes.

The solubilized mixture is then filtered using a mechanical filtering aid at ambient temperature or at 30-45° C. Filter aids are well known in the art and include, but are not limited to, diatomaceous earth (DE) such as Celite® 545, perlite, activated carbon, alumina (basic, acidic, neutral) and cellulose. Filter aids are relatively porous particles that are either filtered as a precoat onto the medium or mixed as a body feed with the suspension. In one embodiment of the invention, DE is used as the filter aid. Diatomaceous earth consists of the fused skeletal remains of diatoms, with as little as 15% of the total volume is solid while the rest is empty space. Such high porosity facilitates liquid flow around the particles and improves the rate of filtration. If added to the mixture, the filter aid is typically added at concentrations of about 1-10% by weight of the mixture and the suspension of the oleoresin and organic solvent is stirred prior to filtration. During the filtration process, the soft and hard waxes in marigold oleoresin are adsorbed onto the filer aid and the acetone solution (first filtrate) contains trans-lutein esters (trans-L-esters) and trans-zeaxanthin esters (trans-Z-esters). The filter aid is then washed with acetone to remove the colored esters, and the filter aid and wax waste materials are discarded or recycled.

The first filtrate from the filtration step which includes the solvent system is stirred until the crystals of lutein esters and zeaxanthin esters form, or for typically about 8-10 hours. To shorten this process, in one embodiment of the invention, the filtrate is seeded with small quantities of purified esters as seeds (at least about 5 g seeds/1 kg of oleoresin or at least about 0.5 wt % seeds). If seeded, carotenoid esters may begin to crystallize within about 1-2 hours upon stirring at ambient temperature.

The crystalline trans-lutein esters and trans-zeaxanthin esters are next collected in a second filtration step and washed with a small amount (about 1 Kg/1 Kg of oleoresin) of the same or a different solvent system as used in the solubilization step. The preferable solvent for washing the crystals is acetone since its residual levels can be easily removed from crystallized esters. Alternatively, the crystals may be washed with C₁-C₃ alcohols such as methanol (MeOH), ethanol (EtOH), 1-propanol (1-PrOH), and/or 2-propanol (2-PrOH). After acetone or alcohol wash, the crystals dry almost instantly, and any residual solvent may be easily removed under high vacuum at ambient or elevated temperature below 55° C. The unique process of the invention provides lutein esters that are 90-95% pure.

The second filtrate obtained contains mostly a mixture of trans- and cis-isomers of lutein esters and zeaxanthin esters. The solvent can be evaporated from this filtrate and recycled. Following solvent evaporation, marigold oleoresin that is obtained from the second filtrate can then be subjected to a saponification process to provide 85-89% pure lutein. Alternatively, the oleoresin can be used without further purification as an additive in animal feed.

The unique process of the invention provides lutein esters in high purity and without the presence of sticky soft and hard waxes. When acetone is used as the solvent in the process, it provides the additional benefits of having a low boiling point and a high vapor pressure that allows it evaporation under high vacuum at ambient or at elevated temperature below 55° C. Further, acetone is an environmentally friendly solvent and has been listed as a class 3 solvent by the US Department of Health & Human Services—FDA with maximum daily exposure of 50 mg/day (5,000 ppm). The use of acetone as a single crystallization solvent that can be readily recovered and recycled is also significantly less costly than recovery of binary solvents that require separation prior to recycling.

A preferred process for commercialization of lutein esters in accordance with the invention is shown in the flow-chart in FIG. 2. This flow chart shows the conversion of 1 Kg of marigold oleoresin to lutein esters. After solubilizing 1 Kg of marigold oleoresin in 3 Kg of acetone at 45° C. and stirring for 30 min at ambient temperature, the mixture is filtered through diatomaceous earth (such as Celite® 545®). Celite® 545 is used in industry for clarification of solutions that contain residual solids or impurities and is quite cheap. The soft and hard waxes in marigold oleoresin are adsorbed onto the Celite® 545 and the acetone solution (First Filtrate) contains lutein esters (94%) and zeaxanthin esters (6%). The Celite® 545 is washed with 3 Kg of acetone to remove the colored esters. The filtrate is then stirred at ambient temperature for 8-10 h during which crystals of lutein esters and zeaxanthin esters form in the acetone solution. This process can be shortened to a few hours by adding 5.0 g of crystals of purified esters as seeds (5 g seeds/1 Kg of oleoresin or 0.5 Wt. %). The crystals are then collected by a second filtration and washed with 1 Kg of acetone. The crystals dry almost instantly and the residual acetone can be easily removed under high vacuum at ambient temperature. This process gives 140 g of trans-lutein esters as the major product and trans-zeaxanthin esters as the minor product (42-44% yield) that are 90-95% pure.

The second filtrate in 7 Kg of acetone contains 160 g of lutein esters that is equivalent to 89 g lutein. At this point acetone is evaporated and recycled. After acetone evaporation, approximately 860 g of marigold oleoresin is obtained that contains 89 g of (trans+cis)-lutein esters and zeaxanthin esters. This oleoresin can be subjected to Kemin's saponification process for preparation of FloraGlo™ lutein. This saponification should give 62.3 g of lutein that is 85-89% pure. Alternatively, the oleoresin can be used without further purification as an additive in animal feed.

An alternative approach to the Flow Chart shown in FIG. 2 is shown in FIG. 3. In this process, 10 kg of marigold oleoresin is stirred with 8 L of acetone and 100 g of diatomaceous earth at 45° C. for 1 h and the suspension is filtered at 45° C. or the suspension is simply decanted and then filtered. The filtration unit is then washed with 40 L of acetone.

The filtrate in 48 L of acetone that contains a mixture of (trans+cis)-lutein esters and 4-6% of (trans+cis)-zeaxanthin esters is stirred for 12 h at ambient temperature to commence crystallization. The crystals are allowed to settle and acetone is decanted. The crystals are stirred with 40 L of acetone and after decanting the acetone, the crystals are filtered. The crystals are then washed with ethanol and acetone to afford a crystalline mixture of 1.3 Kg of 78% pure (trans+cis)-lutein esters and 4-6% of (trans+cis)-zeaxanthin esters in 36% overall yield.

As noted, the process was developed because after evaluating various solvent systems that have been reported in the literature for crystallization of lutein esters, none produced lutein esters in high purity and in all cases the product was sticky due to presence of soft and hard waxes. Acetone was found to be the most practical solvent for crystallization of lutein esters. However, when acetone alone was used without the removal of waxes, the resulting lutein esters were sticky and had a low purity. However, the removal of waxes by adsorption on Celite® 545 provided lutein and zeaxanthin esters in 90-95% purity. Acetone also has a low boiling point and a high vapor pressure that allow its evaporation under high vacuum at ambient temperature. The use of acetone as a single crystallization solvent that can be readily recovered and recycled is also significantly less costly than recovery of binary solvents that require separation prior to recycling.

The only example in which acetone and ketonic solvents such as 2-butanone, 2-pentanone, or their mixtures were used for crystallization of lutein esters has been reported by Kumar⁹. However, according to the Kumar's process acetone was used to solubilize the impurities in marigold oleoresin to afford an enriched concentrate of xanthophyll esters in 46-64% purity in 10-14% yield. As previously mentioned, when acetone was used as the crystallization solvent, lutein and zeaxanthin esters were obtained in 61-66% purity and 37-38% overall yield and crystals were sticky. Although acetone was readily evaporated, the crystallized lutein and zeaxanthin esters contained significant amounts of waxes. Since soft and hard waxes are known to be insoluble in acetone, the disclosed process was developed in which the waxes were precipitated from acetone solution of lutein and zeaxanthin esters and were removed by filtration through diatomaceous earth (Celite® 545). Upon stirring the filtrate at ambient temperature, lutein and zeaxanthin esters crystallized and were collected by filtration in 90-95% purity and 33-44% overall yield. This overall yield was based on the recovery of the total trans- and cis-lutein esters in marigold oleoresin. Since cis-lutein esters and cis-zeaxanthin esters, similar to other carotenoids do not crystallize during crystallization, the yield of this process was also determined based on the recovery of the trans-lutein esters and trans-zeaxanthin esters in the original marigold oleoresin. In this approach, the yields of lutein esters based on the recovery of total trans-lutein and zeaxanthin esters was 46-60%. It is interesting to note that these carotenoid esters dried on the filtration funnel almost instantly and had virtually no wax content that could contribute to stickiness of the crystals. This process is novel due to the removal of waxes by adsorption on diatomaceous earth (Celite® 545) and the high purity of the crystallized lutein and zeaxanthin esters that is required for commercial production of dietary supplements. It is imperative to point out that trans-lutein esters and trans-zeaxanthin esters are soluble in acetone due to the presence of other natural oils in marigold oleoresin and did not immediately crystallized from the acetone solution. However, after removal of waxes, lutein and zeaxanthin fatty acid esters gradually crystallized from acetone after stirring for 8-10 h at ambient temperature.

Another novel aspect of this process is seeding crystallization of lutein and zeaxanthin esters that resulted in rapid crystallization of these esters. As mentioned earlier, in the absence of seeding crystallization, the acetone solution of lutein and zeaxanthin esters had to be stirred at ambient temperature for 8-10 h to commence crystallization. This was most likely due to polymorphism during crystallization of the mixture of lutein and zeaxanthin esters that were esterified with different fatty acids. To control the crystallization process, after removal of waxes by filtration, the filtrates were seeded with small quantities of purified crystalline lutein and zeaxanthin esters. Therefore, when crystallization of lutein and zeaxanthin esters were carried out with seeding on various scales, carotenoid esters consistently crystallized in 1-2 h in 59-60% yield based on trans-(L+Z)-esters and in 90-95% purity. This approach also resulted in a consistent overall yield of 42-44% based on total ester content of crystals and the second filtrate from crystallization. As pointed out earlier, in all of the seeding experiments, the orange crystals of lutein and zeaxanthin esters began to crystallize within the first hour upon stirring at ambient temperature while in the absence of seeds, the crystals appeared very slowly over an extended period of 8-10 h.

The following examples are offered to illustrate but not limit the invention. Thus, it is presented with the understanding that various formulation modifications as well as method of delivery modifications may be made and still are within the spirit of the invention.

In the following Examples, Celite® 545 or diatomaceous earth were obtained from (Sigma-Aldrich cat # 22140) and Fisher Scientific (cat # S25298).

### EXAMPLE 1

### General Procedure for Crystallization of Lutein Esters and Zeaxanthin Esters from Marigold Oleoresin Using Acetone-Diatomaceous Earth (Experiments 1-7)

Marigold oleoresin (10 g) was transferred into a 125 mL beaker and was treated with specified amount of acetone as shown in Table 1. The beaker was covered with aluminum foil and the mixture was heated at 45-50° C. with stirring for 10 min until the oleoresin became soluble. The mixture was allowed to cool down to ambient temperature (23-25° C.) and stirred for 30 min. The mixture was then filtered through specified amount of diatomaceous earth (Table 1) using a Buchner funnel (6 cm i.d., 3.5 cm h) that was connected to vacuum. The filtration flask was placed in an ice bath to prevent evaporation of acetone. The diatomaceous earth (DE) was washed with 30 g of acetone until the dark red color was removed. The filtrate was then stirred at ambient temperature for 8-10h during which the orange crystals of lutein and zeaxanthin fatty acid esters were slowly formed. The crystals of trans-L-esters and trans-Z-esters were filtered and washed with 10 g of acetone. The crystals were dried under high vacuum at ambient temperature (23-25° C.) in a desiccator overnight to afford a mixture of trans-lutein esters and trans-zeaxanthin esters. The total carotenoid contents of the crystallized esters and their purity was measured by UV-Vis after saponification (Table 1). Similarly, the weight of the total (trans+cis)-esters in the filtrate was determined by UV-Vis after saponification. The relative composition of trans-(L+Z)-esters and their cis-isomers was determined by normal phase HPLC after saponification.

TABLE 1
Experimental conditions for crystallization of a mixture of trans-lutein esters
(trans-L-esters) and trans-zeaxanthin esters (trans-Z-esters) from marigold
oleoresin (10 g) using acetone-diatomaceous earth (DE) and the weight and purity
of crystallized esters determined by UV-Vis after saponification.

| | | | Crystals | Filtrate^{a, b} | Total (trans + cis)- |
|---|---|---|---|---|---|
| | Acetone | DE | trans-(L + Z)-Esters, g | (trans + cis)-(L + Z)-Esters, g | Esters |
| Exp. | g | g | % Purity, g | % trans-Isomers, g | g |
| 1 | 50 | 3.0 | 1.50, 85, 1.28 | 1.60, 58, 0.93 | 2.88 |
| 2 | 40 | 3.0 | 1.13, 96, 1.08 | 1.80, 51, 0.92 | 2.88 |
| 3 | 40 | 2.0 | 1.40, 94, 1.32 | 1.87, 54, 1.01 | 3.19 |
| 4 | 40 | 2.0 | 1.29, 87, 1.12 | 1.84, 60, 1.10 | 2.96 |
| 5 | 40 | 1.0 | 1.09, 91, 0.99 | 1.90, 60, 1.14 | 2.89 |
| 6 | 30 | 1.0 | 1.33, 91. 1.21 | 1.67, 58, 0.97 | 2.88 |
| 7 | 20 | 1.0 | 1.43, 92, 1.32 | 1.57, 56, 0.88 | 2.89 |

^{a}The weight of total carotenoids in the filtrate from crystallization was determine by UV-Vis;
^{b}the relative composition of all-trans-lutein esters (trans-L-esters) and all-trans-zeaxanthin esters (trans-Z-esters) and their cis-stereoisomers in the filtrate from crystallization was determined by normal phase HPLC after saponification.

The overall yields and the yields based on recovery of the trans-(L+Z)-esters in crystallization of carotenoid esters from oleoresin on different scales using acetone and diatomaceous earth as adsorbent is shown in Table 2. The data shown in Table 2 were calculated based on the weight and purity of carotenoid esters in crystals and filtrate from crystallization provided in Table 1. The second filtrate also contained significant amounts of (trans+cis)-carotenoid esters. As shown in Table 2, the optimum amount of diatomaceous earth that was needed for the removal of waxes was 1 g/10 g of oleoresin and additional amounts of this adsorbent did not improve the overall yield of carotenoid esters. Lower amounts of diatomaceous earth could also be used but provided L-esters in lower purities ranging from 60-78%.

TABLE 2
The yields of trans-lutein esters and trans-zeaxanthin esters [trans-( L
+ Z)-esters] and overall yields of crystallization of carotenoid
esters from 10 g of oleoresin with acetone using diatomaceous earth (DE) as adsorbent.

| | | Crystals | Filtrate | Yield Based on | Overall Yield Based on |
|---|---|---|---|---|---|
| | | trans-(L + Z)- | trans-(L + Z)- | trans-(L + Z)- | (trans + cis)-(L + Z)- |
| | Acetone, DE | Esters | Esters | Esters^{b} | Esters^{c} |
| Exp. | g, g | g, % L/Z^{a} | g | % | % |
| 1 | 50, 3.0 | 1.28, 95.52/4.48 | 0.93 | 1.28/2.21 = 58 | 1.28/2.98 = 43 |
| 2 | 40, 3.0 | 1.08, 95.75/4.25 | 0.92 | 1.08/2.00 = 54 | 1.08/2.98 = 36 |
| 3 | 40, 2.0 | 1.32, 95.67/4.33 | 1.01 | 1.32/2.33 = 57 | 1.32/2.98 = 44 |
| 4 | 40, 2.0 | 1.12, 95.64/4.36 | 1.10 | 1.12/2.22 = 50 | 1.12/2.98 = 38 |
| 5 | 40, 1.0 | 0.99, 95.53/4.47 | 1.14 | 0.99/2.13 = 46 | 0.99/2.98 = 33 |
| 6 | 30, 1.0 | 1.21, 95.60/4.40 | 0.97 | 1.21/2.18 = 56 | 1.21/2.98 = 41 |
| 7 | 20, 1.0 | 1.32, 95.65/4.35 | 0.88 | 1.32/2.20 = 60 | 1.32/2.98 = 44 |

^{a}The ratio of trans-L-ester/trans-Z-esters was determined after saponification of crystallized esters to L and Z;
^{b}yield was calculated by dividing the weight of isolated trans-(L + Z)-esters with total of trans-(L + Z)-esters in crystals and in the filtrate from crystallization;
^{c}overall yield is based on total carotenoid content of 16.6% of (trans + cis)-(L + Z) in the oleoresin that is equivalent to 1.666 × 1.789 = 2.98 g of (trans + cis)-(L + Z)-esters.

### EXAMPLE 2

### General Procedure for Seeding Crystallization of Lutein Esters and Zeaxanthin Esters from Marigold Oleoresin Using Acetone-Diatomaceous Earth (DE) on Different Scales (Experiments 8-11)

Four separate crystallization experiments with seeding were carried out with 10, 20, 30, and 50 g of marigold oleoresin. In each experiment the appropriate amounts of marigold oleoresin were transferred into a beaker and treated with specified amount of acetone as shown in Table 3. The beaker was covered with aluminum foil and the mixture was heated at 45-50° C. with stirring for 10 min until the oleoresin became soluble. The mixture was allowed to cool down to ambient temperature (23-25° C.) and stirred for 30 min. The mixture was then filtered through specified amount of diatomaceous earth (Table 3) using a Buchner funnel that was connected to vacuum. The filtration flask was placed in an ice bath to prevent evaporation of acetone. The diatomaceous earth was washed with appropriate amount of acetone (Table 3) until the dark red color was removed. The filtrate was treated with appropriate amounts of purified crystalline lutein and zeaxanthin esters as seeds and the mixture was stirred at ambient temperature for 8-10 h. The orange crystals of lutein and zeaxanthin fatty acid esters appeared within the first hour. The orange crystals of trans-L-esters and trans-Z-esters were filtered and washed with appropriate amount of acetone (Table 3) and dried under high vacuum in a desiccator overnight to afford a mixture of trans-lutein esters and trans-zeaxanthin esters. The total carotenoid contents of the crystallized esters and their purity was measured by UV-Vis after saponification (Table 3). Similarly, the weight of the total (trans+cis)-esters in the filtrate was determined by UV-Vis after saponification. The relative composition of trans-(L+Z)-esters and their cis-isomers was determined by normal phase HPLC after saponification.

TABLE 3
Experimental conditions for seeding crystallization of a mixture of trans-lutein
esters (trans-L-esters) and trans-zeaxanthin esters (trans-Z-esters) from
marigold oleoresin using acetone-diatomaceous earth (DE) and the weight and
purity of crystallized esters determined by UV-Vis after saponification.

| | | | Seeding Wt. of | Crystals^{b} | Filtrate^{c, d} | Total |
|---|---|---|---|---|---|---|
| | Oleoresin | | trans-(L + Z)- | trans-(L + Z)- | (trans + cis)-(L + Z)- | [trans + cis)- |
| | Acetone | DE^{a} | Esters | Esters, g | Esters, g | Esters |
| Exp. | g, g | g | g | % Purity, g | % trans-Isomers, g | g |
| 8 | 10, 30 | 1.0 | 0.05 | 1.40, 94, 1.32 | 1.68, 54, 0.91 | 3.00 |
| 9 | 20, 60 | 2.0 | 0.10 | 2.75, 95, 2.61 | 3.36, 52, 1.75 | 5.97 |
| 10 | 30, 90 | 3.0 | 0.15 | 4.05, 92, 3.73 | 5.16, 50, 2.58 | 8.89 |
| 11 | 50, 150 | 5.0 | 0.25 | 6.91, 90, 6.22 | 8.66, 51, 4.42 | 14.88 |

^{a}In experiments 8-11, diatomaceous earth (DE) was washed with 30, 60, 90, and 150 g of acetone, respectively;
^{b}in experiments 8-11 crystals were washed with 10, 20, 30, and 50 g acetone, respectively;
^{c}the weight of total carotenoids in the filtrate from crystallization was determine by UV-Vis;
^{d}the relative composition of all-trans-lutein esters (trans-L-esters) and all-trans-zeaxanthin esters (trans-Z-esters) and their cis-stereoisomers in the filtrate from crystallization was determined by normal phase HPLC after saponification.

The seeding crystallization experiments were carried out in order to optimize the consistency of crystallization and obtain more uniform crystals of lutein and zeaxanthin esters. The overall process for seeding crystallization of lutein and zeaxanthin esters with acetone using Celite® 545 or DE as adsorbent was identical to the process outlined in FIG. 2. However, after filtration of acetone solution of lutein-esters and zeaxanthin-esters through Celite® 545® or DE, the filtrates from experiments 8-11 were treated with appropriate amount of purified crystalline lutein and zeaxanthin esters (Table 3).

The overall yields and the yields based on recovery of the trans-(L+Z)-esters in seeding crystallization of carotenoid esters from oleoresin on different scales employing acetone and diatomaceous earth (Celite® 545) as adsorbent is shown in Table 4. The data shown in Table 4 were calculated based on the weight and purity of carotenoid esters in crystals and filtrate from crystallization provided in Table 3. The second filtrate also contained significant amounts of (trans+cis)-carotenoid esters.

TABLE 4
The yields of trans-lutein esters and trans-zeaxanthin esters [trans-(L +
Z)-esters] based on recovery of trans-esters and overall yields of carotenoid
esters from oleoresin with acetone using diatomaceous earth (DE) as adsorbent.

| | | | Filtrate | Yield Based on | Overall Yield Based on |
|---|---|---|---|---|---|
| | Oleoresin, | Crystals | trans-(L + Z)- | trans-(L + Z)- | (trans + cis)-(L + Z)- |
| | Acetone, DE | trans-(L + Z)-Esters | Esters | Esters^{b} | Esters^{c} |
| Exp. | g, g, g | g, % L/Z^{a} | g | % | % |
| 8 | 10, 30, 1.0 | 1.32, 95.70/4.30 | 0.91 | 1.32/2.23 = 59 | 1.32/3.00 = 44 |
| 9 | 20, 60, 2.0 | 2.61, 95.65/4.35 | 1.75 | 2.61/4.36 = 60 | 2.61/5.97 = 44 |
| 10 | 30, 90, 3.0 | 3.73, 95.67/4.33 | 2.58 | 3.73/6.31 = 59 | 3.73/8.89 = 42 |
| 11 | 50, 150, 5.0 | 6.22, 95.68/4.32 | 4.42 | 6.22/10.64 = 59 | 6.22/14.88 = 42 |

^{a}The ratio of trans-L-ester/trans-Z-esters was determined after saponification of crystallized esters to L and Z;
^{b}yield was calculated by dividing the weight of isolated trans-(L+Z)-esters with total of trans-(L + Z)-esters in crystals and in the filtrate from crystallization;
^{c}Overall yield is based on total (trans + c/s)-(L + Z)-esters in the crystals and filtrate.

### EXAMPLE 3

### General Procedure for Crystallization of Lutein Esters and Zeaxanthin Esters from 100 g of Marigold Oleoresin Using Acetone-Diatomaceous Earth (DE) (Experiments 12)

Marigold oleoresin (100 g) was transferred into a 1 L beaker and was treated with 300 g of acetone. The beaker was equipped with a magnetic stir bar and a temperature probe and placed on a hot plate. The beaker was covered with aluminum foil and was heated to 45- 50° C. until the oleoresin became soluble. The beaker was then stirred at ambient temperature until the solution temperature reached 25° C. The solution of oleoresin in acetone was filtered using a Buchner funnel that was packed with 10 g of diatomaceous earth (DE). The filtration flask was placed in an ice-bath to prevent evaporation of acetone. The diatomaceous earth was washed with 312 g of acetone to remove most of the color. The filtrate (the first filtrate) was stirred at ambient temperature overnight to commence crystallization. The crystalline lutein and zeaxanthin esters were filtered and washed with 100 g of acetone to produce the second filtrate. The wet weight of the orange crystals was 18 g that after drying for two consecutive days at 45° C. in a high vacuum oven were dried reached a constant weight of 14.91 g (92% purity). The crystallization yield was 45%.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

### REFERENCES

1. Khachik F, Beecher G R. Separation and identification of carotenoids and carotenol fatty acid esters in some quash products by liquid chromatography, Part I: Quantification of carotenoids and related esters by HPLC. J. Agric. Food Chem., 1988, 36(5), 929-937.

2. Khachik F, Beecher G R, Lusby W R. Separation and identification of carotenoids and carotenol fatty acid esters in some squash products by liquid chromatography, Part II: Isolation and characterization of carotenoids and related esters. J. Agric. Food Chem., 1988, 36(5), 938-946.

3. Khachik F, Beecher G R. Separation of carotenol fatty acid esters by high performance liquid chromatography. J. Chromatogr., 1988, 449, 119-133

4. Khachik F, Beecher G R, Lusby W R. Separation, identification, quantification of the major carotenoids in extracts of apricots, peaches, cantaloupe, and pink grapefruit by liquid chromatography. J. Agric. Food Chem., 1989, 37(6), 1465-1473.

5. Philip T. Purification of lutein-fatty acid esters from plant materials. U.S. 4,048,203 A, 1977.

6. Levy L W. Trans-xanthophyll ester concentrates of enhanced purity and methods of making same. WO9954408 A1, 1999.

7. Madhavi D L., Kagan D I Process for the preparation of lutein ester concentrate from marigold oleoresin. US20070185342 A1, 2007.

8. Wu W, Bi L, Xu W, Zhao L, Zhang X. Food-grade high-content lutein ester and its preparation method thereof. CN103130699 A, 2013.

9. Sunil Kumar T K. Novel trans-lutein enriched xanthophyll ester concentrate and a process for its preparation. US 20030229142 A1, 2003.

10. Sadano S, Sonoda T. Purification of lutein fatty acid esters. JP2002030068 A, 2002.

11. Sadano S, Fujiwara K, Harada K. Method for the purification of marigold oleoresin. U.S. Pat. No. 7,214,379 B2, 2007.

12. Ornelas-Cravito A, Hernandez-Hernandez E. Enhanced purity trans-lutein-ester composition and methods of making same. U.S. Pat. No. 7,351,424 B2, 2008.

13. Ornelas-Cravito A. Trans-lutein xanthophyll ester of high purity and high bioavailability in micellar solution and a process for the preparation thereof. US 20070065487 A1, 2007.

14. Xu H, Liu Z, Ning F, Shen Y, Jiang B. Purification process for xanthophyll fatty acid esters and xanthophylls by microporous adsorptive resin column chromatography. CN1810785 A, 2006.

15. Chen W, Liu Yuemin, Liu Yongmei. CN106748947 A, 2017.

16. Dai G, Su J, Wang X, Jiang T, OLE M, Yin Y. Method for extracting high purity lutein ester from marigold ointment. CN101891664 A, 2010.

17. Wu W, Huang Y, Xu W, Fan Z, Lv z, Liu Q. Method for preparing composition containing ester. CN 102349647, 2012.

18. Wang J. Method for preparing high-purity lutein ester from pollen granules of Tagetes erecta. CN102757373, 2012.

19. Tan C. High-purity lutein ester composition and preparation process thereof. CN104432003 A, 2015.

20. Peng Q. Method for preparing high-purity xanthophyll ester from xanthophyll extractum. CN104447462 A, 2015.

21. Yao H, Lin J, Xu X, Tao Z. Method for rapidly preparing high-content natural lutein ester. CN107987001 A, 2018.

22. Rao J R, Reddy G B S. Supercritical carbon dioxide extraction of lutein diesters from ground marigold meal. WO2003037833 A1, 2003.

23. Bhartia U S, Show Reddy G B. Single stage lutein ester extraction from tagetes species-marigold flower meal. US 20140035180 A1, 2014.

24. Ning A. Method for producing high-purity lutein ester from marigold extract. CN107987002 A, 2018.

25. Lian Y, An X, Gao W, Wang H, Yang L. Industrial method for preparing high-purity lutein ester. CN109134254 A, 2019.

1. A process for obtaining purified lutein esters and zeaxanthin esters from extracts of marigold flowers and plants comprising:
solubilizing marigold oleoresin in organic solvent to obtain a mixture;
filtering the mixture through a filter aid to form a filtrate; and
stirring the filtrate to form purified crystals of lutein and zeaxanthin esters.

2. The process of claim 1 whereby the marigold oleoresin is solubilized in an organic solvent selected from the group consisting of PrOH/EtOAc, PrOH/acetone, EtOAc/acetone and acetone.

3. The process of claim 2 whereby the organic solvent is acetone.

4. The process of claim 1 whereby the solvent is used in a ratio of about 0.1:10 to about 10:0.1 to marigold oleoresin.

5. The process of claim 1 whereby the marigold oleoresin is solubilized in the organic solvent at a temperature of at least 40° C.

6. The process of claim 1 whereby the mixture is filtered through or stirred with a filter aid selected from the group consisting of diatomaceous earth, perlite, activated carbon, alumina (basic, acidic, neutral) and cellulose.

7. The process of claim 6 whereby the filter aid is diatomaceous earth or Celite® 545.

8. The process of claim 1 whereby waxes in the marigold oleoresin are adsorbed onto the filter aid during stirring and/or the filtering step.

9. The process of claim 1 whereby the crystals are formed by stirring the filtrate for about 8 to 10 hours.

10. The process of claim 1 further including the step of evaporating and recycling the organic solvent from the filtrate.

11. The process of claim 1 further including the step of adding small amounts of crystalline trans-(L+Z)-ester seeds to the filtrate to more rapidly crystalize the lutein and zeaxanthin esters.

12. The process of claim 1 whereby the seeds are added in an amount of about 0.5% to about 1% of the weight of the oleoresin.

13. The process of claim 1 whereby the marigold oleoresin is solubilized by stirring in the organic solvent for at least 30 minutes.

14. The process of claim 13 whereby the marigold oleoresin is stirred in the organic solvent at a temperature of between about 23 to about 25° C.

15. The process of claim 1 whereby the crystalline lutein esters and zeaxanthin esters are washed with alcohols.

16. Process of claim 15 whereby the alcohol is selected from the group consisting of one or more of methanol, ethanol, 1-propanol and 2-propanol.

17. The process of claim 1 further including the step of drying the crystallized lutein and zeaxanthin esters under high vacuum.

18. The process of claim 17 whereby the crystallized lutein and zeaxanthin esters are dried at a temperature of between about 25 to about 45° C.

19. High purity esters of lutein and zeaxanthin formed by the process of claim 1.

20. High purity esters of lutein and zeaxanthin of claim 17 having purity of at least 85%.
